(19) United States
(12) Patent Application Publication
(10) Pub. No.: US 2013/0144268 A1
(43) Pub. Date: Jun. 6, 2013

(54) SEALING VALVE COVER USED IN SPUTUM SUCTION PIPE

(71) Applicant: Jin CHUNG, Taipei (TW)

(72) Inventor: Jin CHUNG, Taipei (TW)

(73) Assignee: PACIFIC HOSPITAL SUPPLY CO., LTD.

(21) Appl. No.: 13/309,861

(22) Filed: Dec. 2, 2011

(51) Int. Cl. A61M 1/00

(52) U.S. Cl. 604/540

(57) ABSTRACT

A sealing vale cover used in a sputum suction pipe, which is mounted in a sputum suction pipe, includes a valve body and an elastic contractible cover. The valve body has a hollow interior and an open end. The elastic contractible cover seals the other end of the valve body. The inner wall surface of the elastic contractible cover is provided with a contact rib. The contact rib extends radially and outwardly from the top of an inner wall surface of the elastic contractible cover. The contact rib is formed with an arc-shaped surface on its protruding portion. When a long conduit of the sputum suction pipe is disposed through the elastic contractible cover, the arc-shaped surface of the contact rib is brought into contact with the outer surface of the long conduit, thereby preventing the leakage of the sputum.

### BACKGROUND OF THE INVENTION

1. Field of the Invention

The present invention relates to a medical equipment, and in particular to a sealing valve cover used in a sputum suction pipe.

2. Description of Prior Art

In general, sputum suction pipes for medical use are classified into two categories: closed type and open type. The closed-type sputum suction pipe sucks the sputum of a patient while maintaining the breath of the patient without removing the respiratory pipes of the patient. Thus, the closed-type sputum suction pipe reduces the influence of sputum suction on the vital signs of the patient, and prevents the respiratory tract infection of the patient during the sputum suction or the cross infection in the hospital. However, the medicament or disinfectant inserted into the sputum suction pipe for cleaning or sterilizing the sputum of the patient may flow into the respiratory tract of the patient to affect his/her breath. Even, a negative pressure device used during the sputum suction may draw out almost of the oxygen contained in the sputum suction pipe, which causes an oxygen deficiency to the patient.

In view of this, the present invention provides a novel and reasonable structure based on his expert knowledge and deliberate researches.

### SUMMARY OF THE INVENTION

The present invention is to provide a sealing valve cover used in a sputum suction pipe, which is mounted in a sputum suction pipe for preventing the medicament or disinfectant injected into the sputum suction pipe from flowing toward the patient and being inhaled by the patient. Further, the sealing valve cover is capable of preventing the oxygen in the sputum suction pipe from being drawn out by a negative pressure device, thereby preventing the oxygen deficiency in the sputum suction pipe.

The present invention provides a sealing vale cover used in a sputum suction pipe, which is mounted in a sputum suction pipe and includes a valve body and an elastic contractible cover. The valve body has a hollow interior and an open end. The elastic contractible cover seals the other end of the valve body. The inner wall surface of the elastic contractible cover is provided with a contact rib. The contact rib extends radially and outwardly from the top of an inner wall surface of the elastic contractible cover. The contact rib is formed with an arc-shaped surface on its protruding portion. When a long conduit of the sputum suction pipe is disposed through the elastic contractible cover, the arc-shaped surface of the contact rib is brought into contact with the outer surface of the long conduit, thereby preventing the leakage of the sputum.

### BRIEF DESCRIPTION OF DRAWING

FIG. 1 is a perspective view showing a first embodiment of the present invention;

FIG. 2 is a perspective view showing the first embodiment of the present invention taken from another viewing angle;

FIG. 3 is a top view showing the first embodiment of the present invention;

FIG. 4 is a cross-sectional view showing the first embodiment of the present invention;

FIG. 5 is a schematic view (I) showing the action of the present invention in the sputum suction pipe;

FIG. 6 is a schematic view (II) showing the action of the present invention in the sputum suction pipe;

FIG. 7 is a schematic view (III) showing the action of the present invention in the sputum suction pipe;

FIG. 8 is a top view showing a second embodiment of the present invention;

FIG. 9 is a top view showing a third embodiment of the present invention;

FIG. 10 is a top view showing a fourth embodiment of the present invention; and

FIG. 11 is a top view showing a fifth embodiment of the present invention.

### DETAILED DESCRIPTION OF THE INVENTION

In order to make the Examiner to better understand the characteristics and technical contents of the present invention, a detailed description relating thereto will be made with reference to the accompanying drawings. However, the drawings are illustrative only, but not used to limit the scope of the present invention.

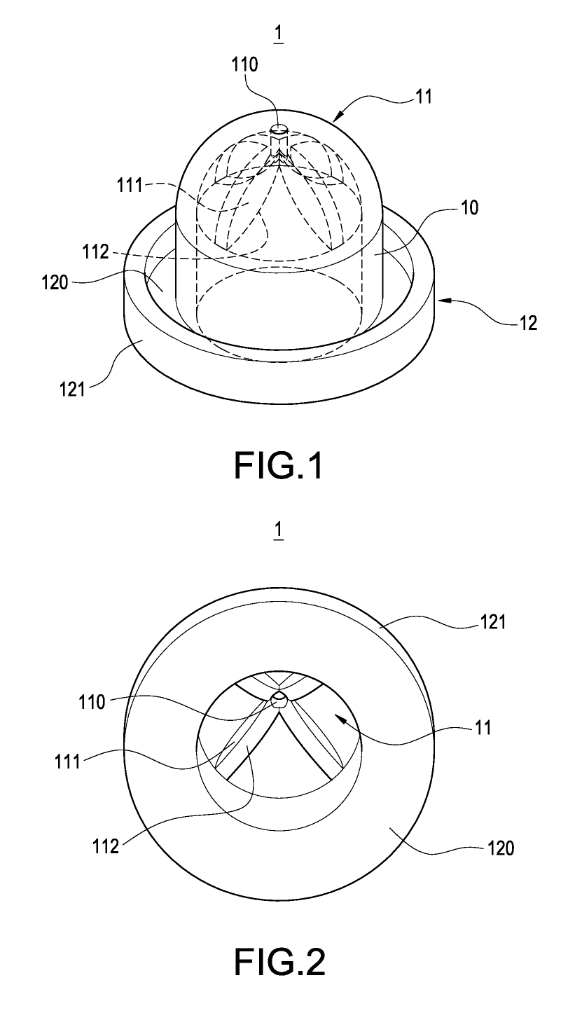

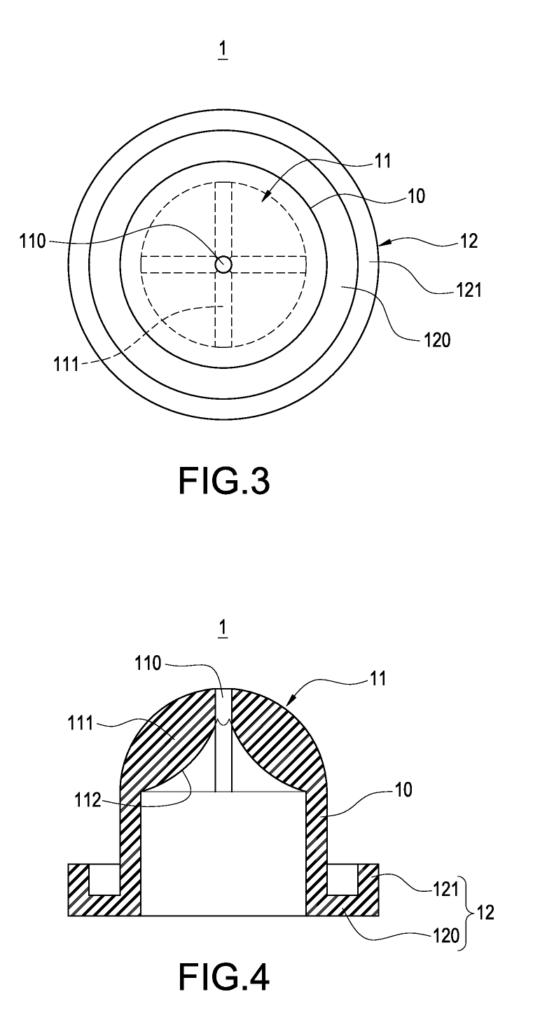

Please refer to FIGS. 1 to 4. FIG. 1 is a perspective view showing the first embodiment of the present invention. FIG. 2 is a perspective view showing the first embodiment of the present invention taken from another viewing angle. FIG. 3 is a top view showing the first embodiment of the present invention. FIG. 4 is a cross-sectional view showing the first embodiment of the present invention. The present invention provides a sealing valve cover used in a sputum valve pipe. The sealing valve cover 1 includes a valve body 10 and an elastic contractible cover 11 located on one end of the valve body 10.

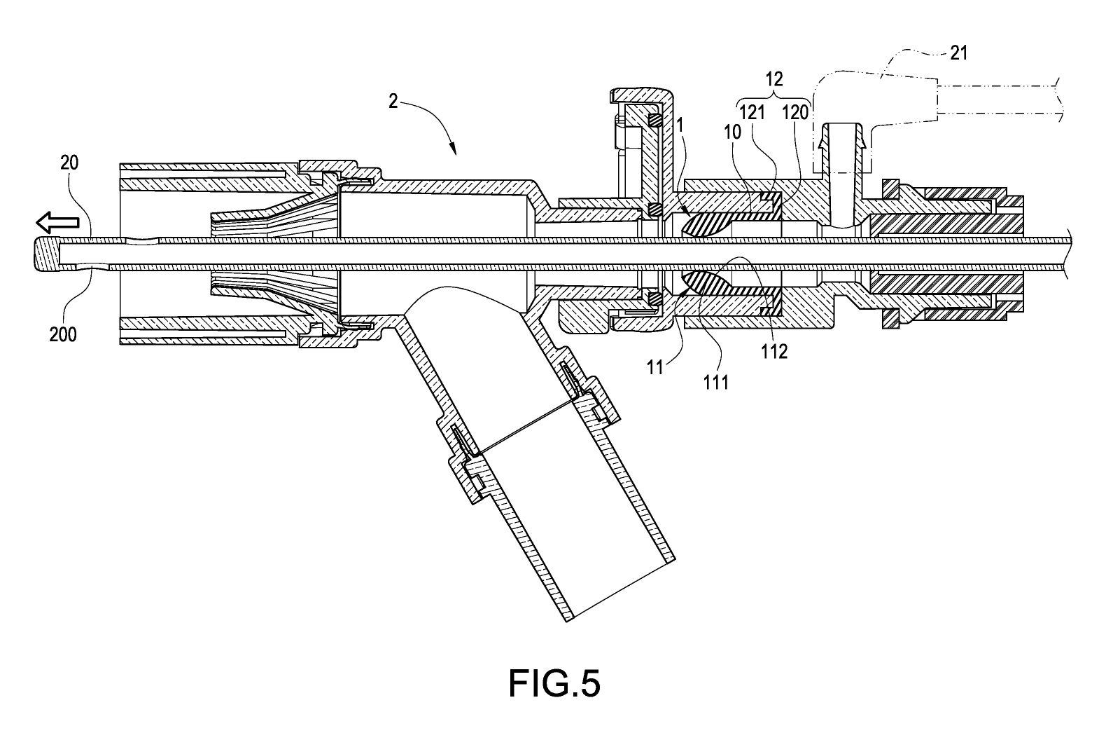

The valve body 10 is mounted in a sputum suction pipe 2 (FIG. 5), so that the outer profile and the dimension of the valve body 10 are required to correspond to the inner diameter and dimension of the sputum suction pipe 2. The valve body 10 has a hollow interior. One end of the valve body 10 allows the elastic contractible cover 11 to be disposed thereon, and the other end of the valve body 10 is an open end for allowing a long conduit 20 of the sputum suction pipe 2 to be disposed through. In order to fix the valve body 10 into the sputum suction pipe 2, an outer edge of the other end of the valve body 10 is provided with a retaining ring 12. The retaining ring 12 comprises an annular edge 120 extending laterally form the outer edge of the other end of the valve body 10, and a flange 121 protruding from the annular edge 120. By this arrangement, the retaining ring 12 can be inserted into the wall surface of the sputum suction pipe 2.

The elastic contractible cover 11 is formed into a cover shape for sealing one end of the valve body 10. The top of an inner surface of the elastic contractible cover 11 is provided with a through-hole 110 approximately located in its center. The long conduit 20 of the sputum suction pipe 2 is disposed through the valve body 10 via the through-hole 110. The elastic contractible cover 11 may be integrally formed on the valve body 10. The inner wall surface of the elastic contractible cover 11 is provided with at least one contact rib 111. There may be a plurality of contact ribs 111 each extending radially and outwardly from the through-hole 110. Each of the contact ribs 111 is formed with an arc-shaped surface 112 on its protruding portion. When the long conduit 20 is disposed through the through-hole 110, the arc-shaped surface 112 of each contact rib 111 is brought into contact with the outer surface of the long conduit 20. In the first embodiment of the present invention, the elastic contractible cover 11 is formed into a semi-arc shape.

By the above-mentioned constituents, the sealing valve cover used in a sputum suction pipe according to the present invention can be obtained.

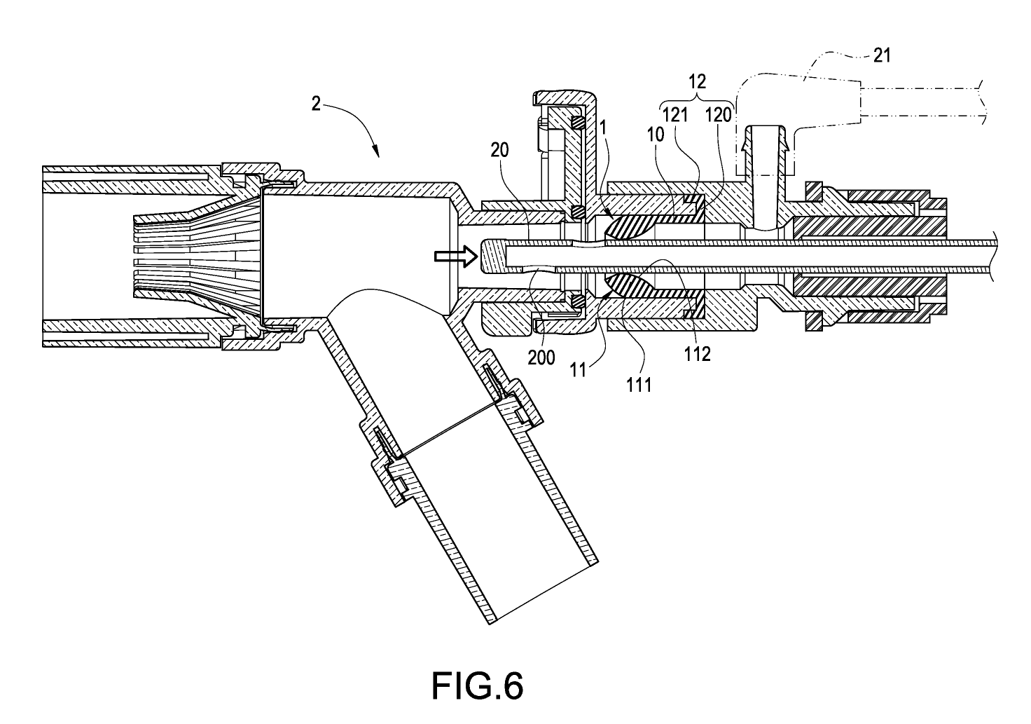

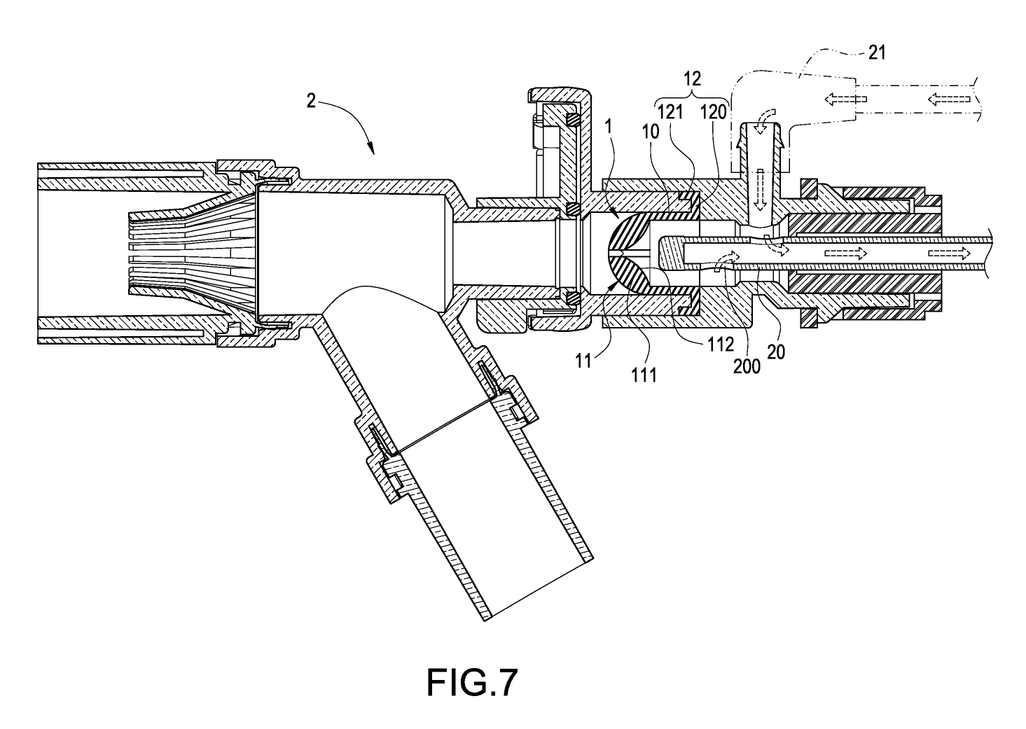

As shown in FIG. 5, when a patient is subjected to a sputum suction process, the long conduit 20 of the sputum suction pipe 2 is disposed into the valve body 10 and through the through-hole 110 of the elastic contractible cover 11. At this time, the contact ribs 111 of the elastic contractible cover 11 are brought into contact with the outer surface of the long conduit 20. By this arrangement, although the long conduit 20 contacts the sputum of the patient, the sputum cannot leak from the elastic contractible cover 11. As shown in FIGS. 6 and 7, when the long conduit 20 is drawn out of the valve body 10 for replacement, although a lot of sputum of the patient is adhered onto the outer surface of the long conduit 20, the sputum is collected in the valve body 10 by the contact ribs 111 of the elastic contractible cover 11, thereby cleaning the long conduit 20 in the valve body 10. As shown in FIG. 7, an injection pipe 21 externally connected to one side of the sputum suction pipe 2 is used to inject a medicament or disinfectant into the valve body 10 to clean or sterilize the end of the long conduit 20 inserted into the valve body 10, thereby preventing the sputum from infecting other patients. The medicament or disinfectant after the cleaning or sterilizing process is introduced into the long conduit 20 via a guiding hole 200 on one end of the long conduit 20, thereby draining the used medicament or disinfectant. The long conduit 20 after the cleaning or sterilizing process can be drawn out of the sputum suction pipe 2. Since the long conduit 20 has been cleaned or sterilized, the cross infection in the hospital can be reduced efficiently. The sealing valve cover 1 can prevent the injected medicament or disinfectant from flowing toward the patient, and also prevent the oxygen in the sputum suction pipe from drawing to the outside via a negative pressure device.

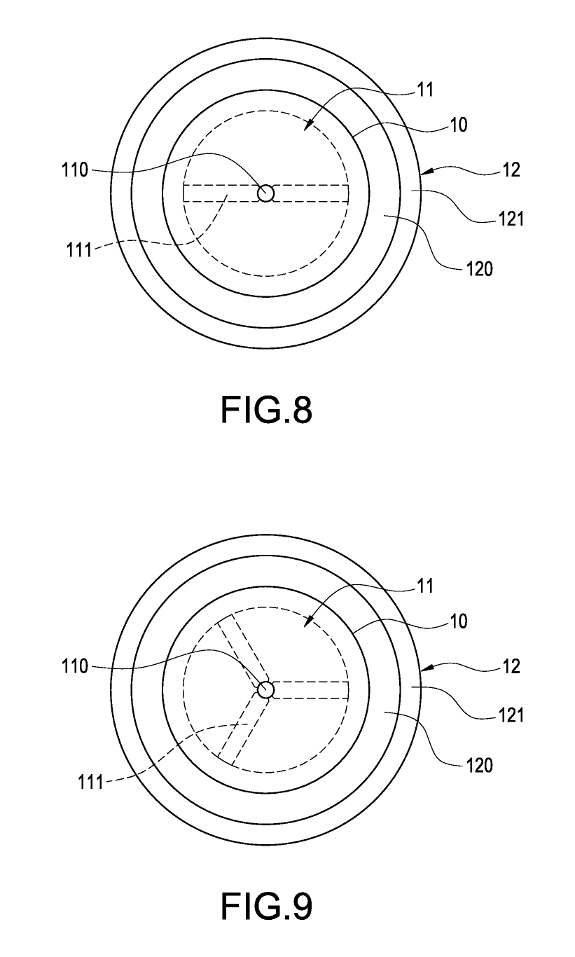

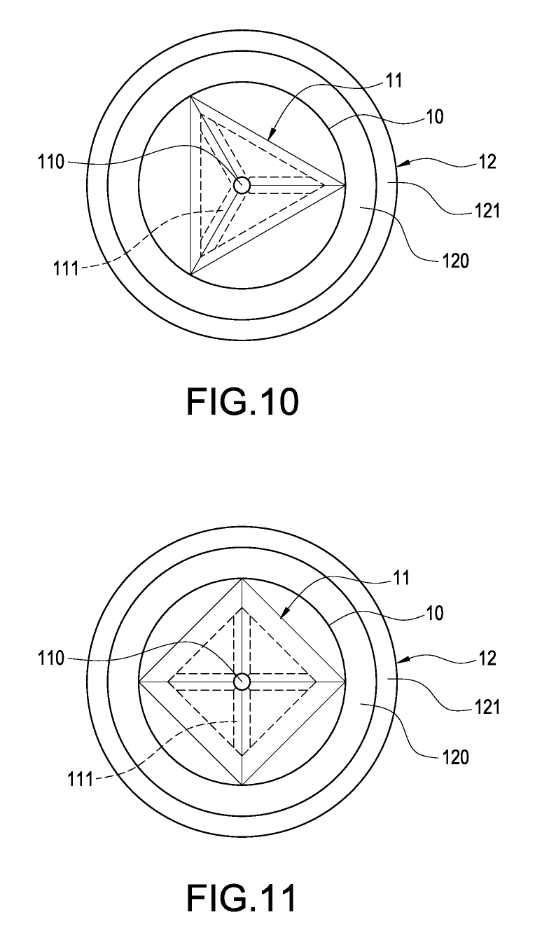

FIG. 8 and FIG. 9 are a top view showing the second embodiment and the third embodiment of the present invention respectively. In the second and third embodiments, the number of the contact ribs 111 of the elastic contractible ribs 11 may be two or three (instead of four in the first embodiment). FIG. 10 and FIG. 11 are a top view showing the fourth embodiment and the fifth embodiment of the present invention respectively. In the fourth and fifth embodiments, the elastic contractible cover 11 may be formed into a triangular pyramid shape or square pyramid shape instead of the semi-arc shape in the first embodiment. Thus, the shape of the elastic contractible cover 11 and the number of the contact ribs 111 are not limited to the above forms.

According to the above, the present invention really solves the problems in prior art and achieves desired objects. Further, the present invention demonstrates novelty and inventive steps, so that the present invention conforms to the requirements for an invention patient.

Although the present invention has been described with reference to the foregoing preferred embodiments, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims.

What is claimed is:

1. A sealing valve cover used in a sputum suction pipe, mounted in a sputum suction pie and including:
a valve body having a hollow interior and an open end; and
an elastic contractible cover sealing the other end of the valve body, an inner wall surface of the elastic contractible valve being provided with a contact rib, the contact rib extending radially and outwardly from the top of the inner wall surface of the elastic contractible cover, the contact rib being provided with an arc-shaped surface on a protruding portion thereof.

2. The sealing valve cover used in a sputum suction pipe according to claim 1, wherein an outer edge of the valve body is provided with a retaining ring for inserting into the sputum suction pipe.

3. The sealing valve cover used in a sputum suction pipe according to claim 2, wherein the retaining ring comprises an annular edge extending laterally from the outer edge of the valve body, and a flange protruding from the annular edge.

4. The sealing valve cover used in a sputum suction pipe according to claim 1, wherein there are a plurality of contact ribs.

5. The sealing valve cover used in a sputum suction pipe according to claim 1, wherein the top of the inner wall surface of the elastic contractible cover is provided with a through-hole.

6. The sealing valve cover used in a sputum suction pipe according to claim 5, wherein the elastic contractible cover is integrally formed on the valve body.

7. The sealing valve cover used in a sputum suction pipe according to claim 5, wherein the elastic contractible cover is formed into a semi-arc shape.

8. The sealing valve cover used in a sputum suction pipe according to claim 5, wherein the elastic contractible cover is formed into a pyramid shape.

9. The sealing valve cover used in a sputum suction pipe according to claim 8, wherein the elastic contractible cover is formed into a triangular pyramid shape or a square pyramid shape.
